(19) United States
(12) Patent Application Publication
(10) Pub. No.: US 2011/0060178 A1
(43) Pub. Date: Mar. 10, 2011

(54) SLURRY COLUMN GASOLINE ALKYLATION USING GAS PHASE OLEFIN INJECTION

(76) Inventors: Douglas A. Nafis, Mt. Prospect, IL (US); Lev Davydov, Northbrook, IL (US); David N. Myers, Hoffman Estates, IL (US); Carl J. Stevens, Lake Forest, IL (US); Daniel N. Myers, Arlington Heights, IL (US)

(21) Appl. No.: 12/554,044

(22) Filed: Sep. 4, 2009

(51) Int. Cl. C07C 2/58; B01J 8/08; B01J 19/00; B01J 38/00

(52) U.S. Cl. 585/720; 585/721; 422/213; 422/198; 422/212

(57) ABSTRACT

Alkylation systems and processes are provided herein that include a slurry reactor. The slurry reactor receives a reactor feed slurry including catalyst and liquid isobutane, a olefin feed, and a circulating reactor vapor stream, where the slurry reactor produces a reactor liquid effluent stream, the reactor liquid effluent stream including catalyst, isobutane, and a liquid alkylate product. The catalyst in the reactor feed slurry can be regenerated catalyst from a catalyst regenerator. The catalyst can be regenerated after being removed from the liquid alkylate product and isobutane in the reactor liquid effluent stream.

### FIELD OF THE INVENTION

The system and process of the present technology relate to alkylation of isobutane with olefin to make alkylate for gasoline blending, and more particularly to alkylation of isobutane with olefin utilizing a slurry reactor.

### DESCRIPTION OF RELATED ART

In alkylation of isobutane with olefin to make alkylate for gasoline blending, the stability of the catalyst and the quality of the alkylate product are strongly influenced by the local olefin concentration at the catalytic site. A very high ratio of paraffin, such as isobutane (iC₄), to olefin, such as butene, is desirable to ensure that hydride transfer of a once-alkylated intermediate to release iso-octane from the catalytic surface occurs preferentially to alkylation with a second olefin. The latter reaction tends to result in the formation of heavier olefinic species that are difficult to remove from the surface of the catalyst, and eventually results in deactivation of the catalyst.

Existing commercial alkylation processes, such as Sulfuric Acid or HF alkylation, tend to operate at a ratio of reboiled isobutane to olefin feed (i/o) of from about 6:1 to about 12:1. One of the limiting factors is the cost of separating the excess isobutane from the alkylate product, which includes costs associated with both the size of the isostripper and the utility requirements to reboil the isobutane overhead. Thus, economic considerations normally limit the amount of reboiled isobutane that is available for olefin dilution. For effective solid catalyst alkylation, a local i/o ratio in excess of 100:1 and preferably in excess of 200:1 to 500:1 is desirable to provide sufficient catalyst stability for an economic operation. To get from an i/o ratio of from about 6:1 to about 12:1 to a more desired i/o ratio, existing alkylation technologies tend to use a combination of reactor effluent recycle and/or multiple reactor stages with separate olefin injection into each stage.

### SUMMARY OF THE INVENTION

The system and process of the present technology relate to alkylation of isobutane with butenes utilizing a slurry reactor in which olefin is injected in a gas stream with gas diluent.

In one aspect, an alkylation system for alkylation of isobutane with olefin to make alkylate for gasoline blending is provided that includes a slurry reactor, a slurry vessel, a catalyst source, and an isostripper. The slurry reactor receives a reactor feed slurry including catalyst and liquid isobutane, an olefin feed that is in a vapor phase or vaporizes upon being injected into the slurry reactor, and a circulating reactor vapor stream. The circulating reactor vapor stream combines with the olefin in the reactor to form a combined reaction vapor. An alkylation reaction occurs in the reactor when the combined reaction vapor contacts the reactor feed slurry, and a reactor liquid effluent stream is produced. The reactor liquid effluent stream includes catalyst, isobutane, and a liquid alkylate product. The slurry vessel provides the reactor feed slurry to the slurry reactor. The catalyst source provides catalyst to the slurry vessel, and the isostripper provides a liquid isobutane feed stream to the slurry vessel.

In another aspect, an alkylation process for alkylation of isobutane with olefin to make alkylate for gasoline blending is provided. The method includes providing a slurry reactor, providing the reactor feed slurry to the slurry reactor from a slurry vessel, providing catalyst to the slurry vessel from a catalyst source, and providing a liquid isobutane feed stream to the slurry vessel from an isostripper. The slurry reactor receives a reactor feed slurry including catalyst and liquid isobutane, an olefin feed that is in a vapor phase or vaporizes upon being injected into the slurry reactor, and a circulating reactor vapor stream. The circulating reactor vapor stream combines with the olefin in the reactor to form a combined reaction vapor. An alkylation reaction occurs in the reactor when the combined reaction vapor contacts the reactor feed slurry, and a reactor liquid effluent stream is produced.

### BRIEF DESCRIPTION OF THE DRAWINGS

Specific examples have been chosen for purposes of illustration and description, and are shown in the accompanying drawings, forming a part of the specification.

FIG. 1 illustrates one example of an alkylation system of the present technology including a slurry reactor.

FIG. 2 illustrates an enlarged view of the upper portion of the slurry reactor of FIG. 1.

FIG. 3 illustrates an alternative flow scheme for the alkylation system of FIGS. 1 and 2 that includes an inter-cooler solids recycle loop.

### DETAILED DESCRIPTION

The systems and processes disclosed herein conduct alkylation of isobutane with olefin, such as butene, utilizing a slurry reactor. The i/o ratio can be regulated and controlled during the alkylation reaction by supplying olefin at a rate that is less than the rate of reaction. In this manner the olefin concentration can remain depleted at the active sites of the catalyst. In the systems and processes disclosed herein, the olefin feed is in a vapor phase in the slurry reactor. The olefin can be injected into the reactor in a vapor stream, or can be injected into the slurry reactor as a liquid that vaporizes upon being injected into the slurry reactor. The olefin concentration can be controlled by diffusion resistance provided by an induced gas-liquid interface in the reactor. The olefin can be supplied in a manner that controls the diffusion of olefin into the liquid phase based upon the gas phase olefin concentration, bubble size and interfacial area.

It has been found experimentally that a physical diffusion barrier that provides diffusion resistance, and thus a reduction of olefin concentration at the active site of the catalyst, can occur indirectly during catalyst deactivation. An individual catalyst pellet deactivates from the outside to the inside in a “shrinking active core” mode. The deactivated outer layer provides a physical diffusion barrier that slows down the diffusion of olefin to the remaining active interior sites. The physical diffusion barrier helps to keep a lower olefin concentration at the interior sites and those interior sites deactivate less rapidly. Without being bound by any particular theory, it is believed that this phenomenon contributes to the observation that larger catalyst particles, while initially less active than small particles, experience a slower rate of deactivation and have a longer useful life before regeneration becomes necessary.

When such a physical diffusion barrier is allowed to happen naturally by deactivation of the outer catalyst layer, a portion of the catalyst becomes sacrificial, and feed must pass over already deactivated catalyst which tends to result in additional non-beneficial oligomerization. The overall reaction rate tends to become constrained by the diffusion rate because the physical diffusion barrier reduces both the rates of olefin diffusion into the catalyst and the diffusion of alkylate product out of the catalyst. More catalyst volume is ultimately needed to accomplish the same amount of reaction. Additionally, reduction of the diffusion of alkylate product out of the catalyst can result in increased cracking to lights and isomerization to lower TMP/DMH ratio. In contrast, diffusion resistance provided by the induced gas-liquid interface in the reactors of the present systems and processes results in control and limitation of the olefin diffusion rate to the catalyst, but the diffusion of alkylate from the catalyst to the bulk liquid is not affected.

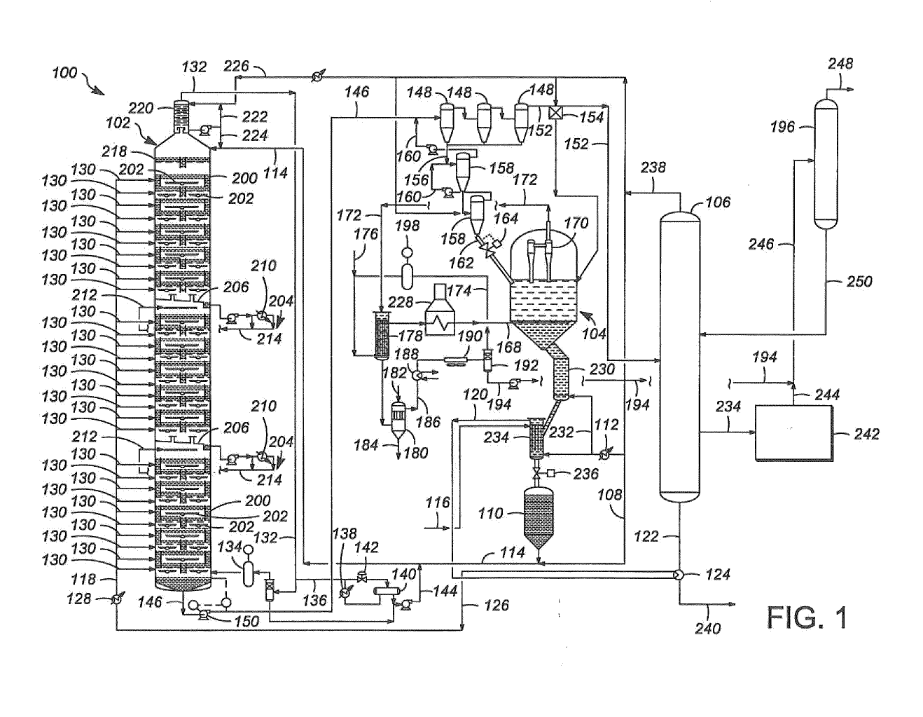

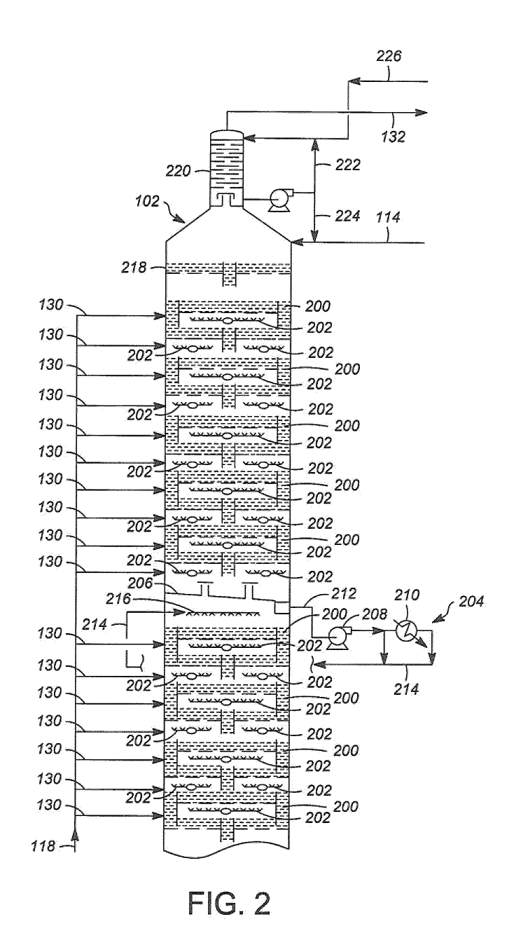

One example of an alkylation system 100 utilizing a slurry reactor is illustrated in FIGS. 1 and 2. Alkylation system 100 includes a slurry reactor 102, a catalyst source, such as catalyst regenerator 104, an isostripper 106, and a slurry vessel 110. The slurry reactor 102 receives a reactor feed slurry including catalyst and liquid isobutane, an olefin feed, and a circulating reactor vapor stream 132, and produces a slurry reactor effluent stream including alkylate product. The slurry vessel 110 provides the reactor slurry to the slurry reactor. The catalyst source provides the catalyst to the slurry vessel, and the isostripper provides an isobutane feed stream to the slurry vessel.

As illustrated in FIGS. 1 and 2, catalyst from the catalyst regenerator 104 and a liquid isobutane feed stream 108 from the isostripper 106 can be provided to a slurry vessel 110. The catalyst can be fresh catalyst, regenerated catalyst, or a combination of fresh and regenerated catalyst. The catalyst can be a solid catalyst, and can be a fine particle catalyst having a nominal diameter from about 20 to about 200 microns and preferably from about 50 microns to about 100 microns. The catalyst can be produced by spray-drying, or by any other suitable means. In one example, the catalyst and the isobutane feed stream 108 can be mixed or otherwise combined at the base of the slurry vessel 110 via a special eduction device to form a reactor feed slurry 114. Reactor feed slurry 114 can contain catalyst in an amount from about 2% by weight of the slurry to about 25% by weight of the slurry, and preferably from about 5% to about 15% by weight of the slurry. The reactor feed slurry 114 can be fed to the top of the slurry reactor 102, and can be provided to the slurry reactor 102 through an inlet.

In the example illustrated in FIGS. 1 and 2, the slurry reactor 102 includes a plurality of distillation trays 200 and a plurality of distributors 202. The distillation trays 200 should be suitable for slurry service and can be for example sieve trays. The plurality of distillation trays can have space between each tray. The slurry reactor 102 can have any suitable dimensions, and can include any suitable number of distillation trays 200. Additionally, the distillation trays 200 can be arranged in any suitable configuration. In one example, the slurry reactor 102 is configured to prevent stagnant areas within the slurry reactor 102 where the solid catalyst can settle out of the reactor slurry 114. As illustrated in FIG. 1, the slurry reactor includes 30 two-pass distillation trays 200, and the main pipe of each of the distributors 202 runs parallel to the downcomers of the two-pass distillation trays 200. Truncated downcomers can be utilized to prevent a stagnant area at the bottom of the downcomer of each distillation tray 200.

Referring back to FIGS. 1 and 2, the reactor feed slurry 114 enters slurry reactor 102 and flows downwardly through the plurality of distillation trays 200, counter-current to a circulating reactor vapor stream 132 that is introduced at the bottom of the slurry reactor 102. The circulating reactor vapor stream flows upwardly through the slurry reactor 102, and is removed from the top of the slurry reactor 102. The circulating reactor vapor stream 132 can act as a diluent gas within the slurry reactor 102.

An olefin feed stream 118 can be provided to the slurry reactor 102. Olefin feed stream 118 can include any suitable olefins, such as butene. Olefin feed stream 118 can also contain saturates such as isobutane (iC₄) and n-butane (nC₄), and limited amounts of propane (C₃) or isopentane (iC₅). The olefin feed stream 118 can include olefins and saturates in an amount sufficient to supply the stoichiometric isobutane needed for alkylation. Olefin feed stream 118 can be divided into a plurality of olefin injection streams 130. The olefin injection streams 130 can be injected into the space between the plurality of distillation trays 200 through a plurality of distributors 202.

The olefin feed stream 118 can be a liquid or a vapor when it is injected into the slurry reactor 102. For example, the olefin feed stream 118 can be a liquid feed stream, and spray nozzles can be utilized on the distributors 202 to introduce the olefin feed as a fine liquid spray that vaporizes in the slurry reactor 102. This example can utilize excess heat of reaction to vaporize the olefin feed, and can reduce the amount of heat utilized to vaporize the olefin external to the slurry reactor 102. In another example, as illustrated in FIG. 1, olefin feed stream 118 can be a vapor stream that is formed from a liquid olefin stream 116. Thus, olefin feed stream 118 can be injected into the slurry reactor 102 as a vapor olefin feed stream. As illustrated, the liquid olefin stream 116 can be provided from a treating unit (not shown), and can be heated in a first heat exchange with hot catalyst in a catalyst cooler 234 to form a first heated olefin stream 120. First heated olefin stream 120 can be further heated in a second heat exchange with isostripper bottoms stream 122 in an alkylate cooler 124 to form a second heated olefin stream 126. Second heated olefin stream 126 can then be passed to a vaporizer 128 that vaporizes the olefin stream to produce olefin feed stream 118.

Referring back to FIGS. 1 and 2, each distributor 202 can be located in a space between the distillation trays 200, and can be below at least a first distillation tray 200 and above at least a second distillation tray 200. Once ejected from a distributor 202 into the space below a first distillation tray 200 and above second distillation tray 200, the olefin injection stream 130 either is a vapor or becomes a vapor upon entering the slurry reactor 102. The circulating reactor vapor stream 132 combines with the olefin vapor of the olefin injection stream 130 in the reactor to form a combined reaction vapor. In one example, the circulating reactor vapor stream 132 has a circulation rate that is high enough to dilute the vapor olefin ejected into the slurry reactor 102 by one or more orders of magnitude.

The combined reaction vapor bubbles up through the first distillation tray 200, which is located above the distributor 202 through which the vapor olefin ejection stream 130 was ejected into the slurry reactor 102, where it comes in contact with the reactor slurry on the first distillation tray 200. An alkylation reaction occurs on the first distillation tray 200 wherein olefin from the combined reaction vapor diffuses into the reactor slurry, reacts with the liquid isobutane in the presence of the catalyst, and produces a liquid phase alkylate product. In order to promote the alkylation reaction, the slurry reactor 102 can operate at a reaction temperature of from about 40 to 120° C. and preferably from about 60° C. to about 80° C. The slurry reactor can also operate at a bubble point pressure corresponding to such a reaction temperature, which can be at about 160 psig.

The rate of diffusion of the olefin out of the combined reaction vapor into the reactor slurry can be maintained at a desired rate that can be lower, and is preferably only slightly lower, than the rate of olefin reaction during alkylation. Maintaining the desired rate of olefin diffusion can be accomplished by controlling the interfacial area between the combined reaction vapor and reactor slurry based on bubble size, gas rate, and tray geometry. Without being bound by any particular theory, it is believed that the olefin concentration in the reactor slurry at the catalyst surface can be maintained at a level low enough to minimize deactivating oligomerization reactions.

The liquid alkylate product is carried downward through the slurry reactor 102 with the downward flowing reactor slurry, forming a reactor liquid that contains both the reactor slurry and the liquid alkylate product. The concentration of liquid alkylate product in the reactor liquid increases on each successive tray downwardly through the slurry reactor 102, and reaches a maximum value at the bottom of the slurry reactor 102. In one example, the amount of reactor slurry 114 provided to the slurry reactor 102 is sufficient to provide an amount of reactor slurry 114 in the reactor liquid at the bottom of the slurry reactor 102 that will prevent the maximum concentration of liquid alkylate product in the reactor liquid at the bottom of the reactor from building up to more than a threshold value. The threshold value of liquid alkylate product in the reactor liquid at the bottom of the reactor can be the amount of liquid alkylate product to which the catalyst can be exposed during the alkylation reaction before alkylate selectivity to undesirable C₉+ products begins to hurt product quality and result in rapid catalyst deactivation. For example, when the catalyst is a Y type zeolite catalyst, the threshold value of liquid alkylate product can be about 15% by weight of the reactor liquid. In examples with some other types of catalyst, the threshold value of alkylate product can be up to about 25% by weight to about 30% by weight of the reactor liquid.

The slurry reactor 102 can also include one or more inter-cooling loops 204, which can remove heat of alkylation reaction from the slurry reactor 102 by removing reactor liquid from the slurry reactor, cooling it, and returning it to the slurry reactor 102. An inter-cooling loop 204 can include a draw-off tray 206, at least one pump 208, and at least one cooler 210. The draw-off tray 206 is located within the slurry reactor 102, and collects reactor liquid as it flows downwardly through the slurry reactor 102. The draw-off tray 206 can be sloped, which can direct collected reactor liquid to an outlet where a draw-off stream 212 can be removed from the slurry reactor 102. The at least one pump 208 can pass the draw-off stream 212 to the at least one cooler 210 to form a cooled draw-off stream 214. The cooled draw-off stream 214 can be injected back into the slurry reactor 102 through a draw-off stream distributor 216. The draw-off stream distributor 216 can be located in the slurry reactor 102 below the draw-off tray 206. Alternatively, cooling for the slurry reactor can also be supplied by other suitable means, such as, for example, a stabbed in heat exchanger.

As the reactor liquid flows downwardly to the bottom of the slurry reactor 102, the gases within the slurry reactor 102 flow upwardly to the top of the slurry reactor 102. A reactor vapor stream 132 can be removed from the top of the slurry reactor 102. Prior to being removed from the top of the slurry reactor 102, the reactor vapor stream 132 can pass through a finishing tray 218, in order to facilitate complete conversion of the vapor olefin injections streams 130. The reactor vapor stream 132 can also pass through a wash section 220 at the top of the slurry reactor 102, which can remove any entrained solids. Wash section 220 can be a disk-and-donut style wash section. Solids entrained in the reactor vapor stream 132 can be washed out of the gas in the wash section 220 by a recirculating stream of isobutane 222, and can be returned to the reactor in a solids return stream 224. An additive isobutane stream 226 can also be added to the recirculating stream of isobutane 222 from the isostripper 106. The reactor vapor stream 132 can be recycled to the bottom of the slurry reactor 102. For example, the reactor vapor stream can be provided to the bottom of the slurry reactor through a compressor 134.

The volume and pressure of the circulating reactor vapor stream 132 can be regulated by separating an excess portion 136 from the circulating reactor vapor stream 132, condensing the excess portion 136 in a condenser 138 to form a condensate to a receiver 140. A hot vapor bypass 142 an also be provided to maintain an interface in the receiver 140. A condensate stream 144 can be provided from the receiver 140, combined with the reactor slurry 114, and returned to the slurry reactor 102.

Alkylation system 100 can also provide for separating the catalyst in the reactor liquid from the liquid alkylate product, and regenerating the catalyst before the catalyst is reintroduced to the slurry reactor 102 in reactor slurry 114. The reactor liquid can be removed from the bottom of the slurry reactor 102 in a reactor liquid effluent stream 146. The reactor liquid effluent stream 146 can contain solid catalyst, liquid alkylate product, and isobutane. The reactor liquid effluent stream 146 can be provided to a at least one first stage liquid solid separation device. As shown in the illustrated embodiment, the at least one first stage liquid solid separation device can be hydroclone 148. It should be noted that any of the hydroclones disclosed herein with respect to the illustrated example could be replaced with other liquid solid separation devices if desired.

In an alternative example, a cold regeneration section (not shown) can be included between the slurry reactor 102 and the at least one first stage hydroclone 148, which can reduce the amount of solids load on the catalyst regenerator 104, which can be a hot catalyst regenerator. The cold regeneration section can be a liquid phase regenerator using hydrogen-saturated isobutane. In this alternative example, a smaller slipstream of catalyst could be sent to the catalyst regenerator 104. Such an example can reduce the energy requirements for heating and cooling of the catalyst between the alkylation reaction temperature, which can be from about 60° C. to about 80° C., to the hot regeneration temperature, which can be from about 250° C. to about 400° C., and could also reduce the heat of vaporization of residual liquid left in the catalyst pores after the hydroclone separation. However, there would likely be an increased usage of isobutane for both the cold regeneration and a flush to remove residual hydrogen.

Referring back to FIGS. 1 and 2, the reactor liquid effluent stream 146 can be provided to the at least one hydroclone 148, by utilizing, for example, at least one slurry pump 150. The slurry pump 150 can be selected to minimize catalyst attrition, and can, for example, have an axial design rather than centrifugal design, a minimal tip speed, and a maximum open area. One example of a pump that can be utilized as a slurry pump 150 is a VTP/VLT vertical, double casing, multi-stage process pump. Without being bound by any particular theory, it is believed that catalyst attrition can be reduced or minimized by keeping the reactor slurry sufficiently dilute and choosing pump designs that minimize shear rates and direction changes in the catalyst flowstream. In an alternative example, the reactor liquid effluent stream 146 can be removed from the slurry reactor 102 at a pressure sufficient to allow the reactor liquid effluent stream 146 to flow through a line to the at least one hydroclone 148 without pumping. In one such example, propane can be used in the recirculating vapor stream 132.

The at least one first stage hydroclone 148 can separate the solid catalyst from the liquid alkylate product and isobutane in the reactor liquid effluent stream 146. As illustrated in FIG. 1, separating solid catalyst from the liquid alkylate product and isobutane in the reactor liquid effluent stream 146 can be performed by a series of hydroclones 148. It has been estimated that, with typical hydroclone efficiencies of about 90%, three stages of hydroclones 148 can reduce the solids content of the hydroclone effluent stream 152 to less than about 50 ppm. Solids and fines remaining in the hydroclone effluent stream 152 can be removed in a filter system 154. Solids and fines removed from the hydroclone effluent stream 152 in the filter system 154 can be back-washed into the catalyst regenerator 104. The hydroclone effluent stream 152 can be provided to the isostripper 106, where C₅+ alkylate products can be separated from nC₄, iC₄ and lighter components that can be in the hydroclone effluent stream 152. An isostripper bottoms stream 122 can be cooled by heat exchange with the olefin feed stream in the alkylate cooler 124 to form a heavy alkylate product stream 240. Isobutane can be removed from the isostripper in the isostripper overhead stream 238.

As illustrated in FIG. 1, a concentrated solids stream 156 from the at least one hydroclone 148 can be provided to at least one second stage hydroclone 158. The concentrated solids stream 156 can be washed with clean, reboiled isobutane in the second stage hydroclone 158, which can remove alkylate products from the pores of the catalyst so that it is not carried into the regenerator 104 with the catalyst. As illustrated in FIG. 1, alkylation system 100 includes two second stage hydroclones 158, which are counter-current hydroclones to minimize isobutane use. The second stage hydroclone overflow streams 160 can be sequentially pumped back to the first stage hydroclones 148 using, for example, at least one diaphragm slurry transport pump or liquid jet eductor pump to minimize catalyst attrition.

A washed concentrated solids stream 162 can be provided from the at least one second stage hydroclone 158 to catalyst regenerator 104 through a slide-valve 164. Catalyst regenerator can be a fluidized bed regenerator. The washed concentrated solids stream 162 can contain isobutane, which can be desorbed from the pores of the catalyst in catalyst regenerator 104 to regenerate the catalyst. In the catalyst regenerator 104, the catalyst can be kept fluidized by a stream of regeneration gas 168, which can be hydrogen. In one example, the catalyst regenerator 104 can provide for a 30 minute catalyst residence time in the catalyst regenerator 104, and can operate at a temperature of about 400° C. and a pressure of about 200 psig.

Regenerator gas effluent stream 172 can pass through one or more cyclones 170 to remove entrained catalyst fines, and can exit the catalyst regenerator 104. Heat can be recovered from the regenerator gas effluent stream 172 by heat exchange of the regenerator gas effluent stream 172 with a recycle gas 174 and makeup hydrogen 176 in a regenerator gas heat exchanger 178. For example, the regenerator gas effluent stream 172 can pass downward through a tube side of the regenerator gas heat exchanger 178, and can be passed to a barrier filter 180 that can remove catalyst fines. The catalyst fines can be periodically removed from the barrier filter 180 in a dry fines stream 184 by using a small hydrogen blowback stream 182.

A filtered regenerator gas stream 186 can be removed from the barrier filter 180, and can be cooled in an isostripper auxiliary reboiler 188 and an air-trim cooler 190. Cooling the filtered regenerator gas stream 186 can cause condensation of heavier hydrocarbons that may be desorbed or cracked off the catalyst during catalyst regeneration, such as C₅+ hydrocarbons. The condensed heavier hydrocarbons can be removed in a knock out (KO) drum 192, and a condensed heavier hydrocarbon stream 194 can be provided to a Butamer section stabilizer 196, along with a reactor effluent stream 244 from a Butamer section reactor 242, in a Butamer section stabilizer feed stream 246. A Butamer section stabilizer overhead stream 248 can contain C₃− that can be utilized for fuel gas. A Butamer section stabilizer bottoms stream 250 can be returned to the isostripper 106. Alternatively, a portion of the Butamer section stabilizer bottoms stream 250 can be provided to the reactor effluent hydroclones 148 to function as wash liquid and reactor effluent filtration backwash liquid to 154, which can reduce isostripper reboiled i/o, as well as the associated capital and utility costs of the isostripper 106.

The gas stream from the KO drum 192 can be used as recycle gas 174. A compressor 198 can be used to provide the recycle gas 174 to the regenerator gas heat exchanger 178. The recycle gas 174 can be further provided from the regenerator gas heat exchanger 178 to a furnace 228 to be heated to regeneration conditions prior to being provided to the catalyst regenerator as regeneration gas stream 168.

Referring back to the catalyst regenerator 104, dried regenerated catalyst can flow from the catalyst regenerator 104 to a catalyst stripper 230. In catalyst stripper 230, a stripping stream 232 of isobutane can strip hydrogen out of the pores of the catalyst, so that it is not carried back to the reactor section where hydrogen could potentially saturate the olefin feed, leading to yield loss. The regenerated catalyst can then be passed to the catalyst cooler 234, where heat can be recovered from the regenerated catalyst by exchange with the olefin feed stream 116. Cooled regenerated catalyst can then be provided to the slurry vessel 110, and can be provided back to the slurry reactor 102 in reactor slurry 114. Flow of the catalyst from the catalyst cooler 234 to the slurry vessel 110 can be regulated by a side valve 236, which sets the overall catalyst circulation rate.

As discussed above, in the illustrated example, the olefin feed stream 116 can contain olefins and saturates such as iC₄ and nC₄ in an amount sufficient to supply the stoichiometric isobutane needs for alkylation. In an alternative example, a saturate feed stream can be provided separate from the olefin feed stream 116. In such an example, a saturate feed stream can be provided to the isostripper 106. The saturate feed stream can be up to about 10% by weight of the total reboiled isobutane removed from the isostripper 106 in isostripper overhead stream 238. To keep n-butane from building up in the isobutane loop, a separate n-butane draw 234 can be taken from the isostripper 106. The n-butane draw 234 can optionally be sent to the Butamer section reactor 242 where the normal butane can be isomerized to an equilibrium mixture of iC₄ and nC₄.

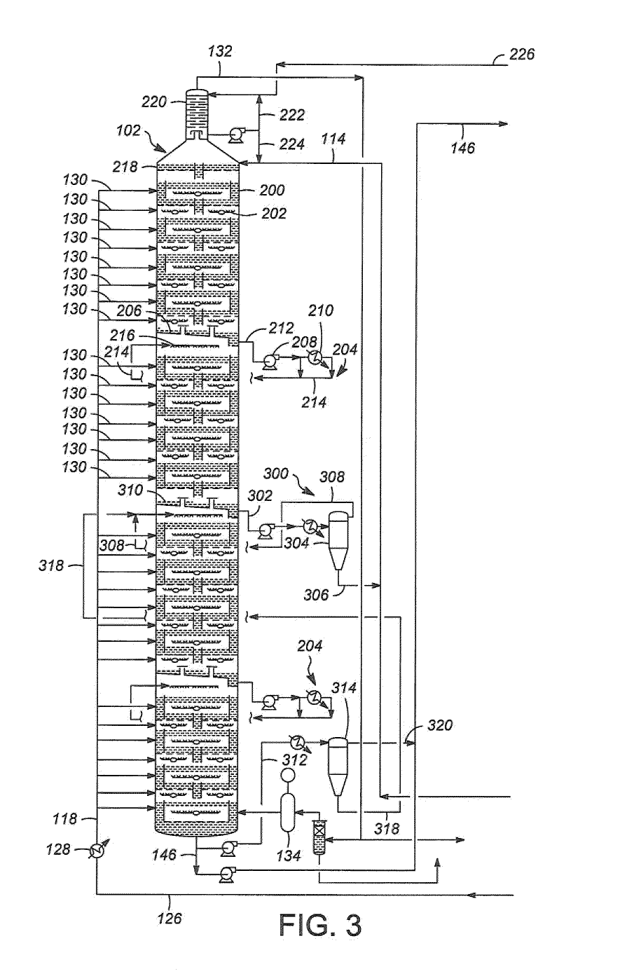

FIG. 3 illustrates an alternative flow scheme for alkylation system 100 that includes an inter-cooler solids recycle loop 300, which provides for the recycle of a portion of solids from the first stage hydroclones 148 to increase the effective c/o without changing the load on the catalyst regenerator 104. This approach can be used, for example, when the slurry reactor 102 is scaled up to capacities higher than about 8000 BPSD of alkylate. As illustrated in FIG. 3, an inter-cooler draw stream 302 can be removed from the slurry reactor and passed to inter-cooler solids recycle loop 300. In the illustrated example, the to inter-cooler solids recycle loop 300 is located at or near the middle of the slurry reactor 102. The inter-cooler draw stream 302 is sent to an inter-cooler hydroclone 304. An inter-cooler concentrated solids stream 306 can be removed from the bottom of the inter-cooler hydroclone 304. The inter-cooler concentrated solids stream 306 can be mixed into the reactor slurry feed stream 114, and can thus be recycled back to the top of the slurry reactor 102. An inter-cooler hydroclone overhead stream 308 can be returned to the slurry reactor returned just below the draw tray 310.

As further illustrated in FIG. 3, a slipstream 312 of the reactor liquid effluent stream 146 can be provided to a slipstream hydroclone 314. A slipstream concentrated solids stream 318 from the bottom of the slipstream hydroclone 314 can be combined with the inter-cooler hydroclone overhead stream 308 and recycled to the slurry reactor 102 just below the draw tray 310. A slipstream hydroclone overhead stream can be combined with the reactor liquid effluent stream 146.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. An alkylation system for alkylation of isobutane with olefin to make alkylate for gasoline blending, the alkylation system comprising:
a slurry reactor that receives a reactor feed slurry including catalyst and liquid isobutane, an olefin feed that is in a vapor phase or vaporizes upon being injected into the slurry reactor, and a circulating reactor vapor stream that combines with the olefin feed in the reactor to form a combined reaction vapor, where an alkylation reaction occurs in the reactor when the combined reaction vapor contacts the reactor feed slurry, and a reactor liquid effluent stream is produced, the reactor liquid effluent stream including catalyst, isobutane, and a liquid alkylate product;
a slurry vessel that provides the reactor feed slurry to the slurry reactor;
a catalyst source that provides catalyst to the slurry vessel; and
an isostripper that provides a liquid isobutane feed stream to the slurry vessel.

2. The alkylation system of claim 1, wherein the catalyst source is a catalyst regenerator.

3. The alkylation system of claim 2, wherein the catalyst in the reactor slurry comprises regenerated catalyst from the catalyst regenerator.

4. The alkylation system of claim 1, wherein the reactor feed slurry comprises catalyst in an amount from about 2% by weight of the slurry to about 25% by weight of the slurry.

5. The alkylation system of claim 1, wherein the slurry reactor comprises a plurality of distillation trays having space between each tray, and a plurality of distributors located in a space below at least a first distillation tray and above at least a second distillation tray, wherein the olefin feed is divided into a plurality of olefin injection streams that are injected into the slurry reactor through the distributors.

6. The alkylation system of claim 5, wherein the combined reaction vapor bubbles up through the first distillation tray, and the alkylation reaction occurs on the first distillation tray wherein olefin from the combined reaction vapor diffuses into the reactor slurry, reacts with the liquid isobutane in the presence of the catalyst, and produces a liquid phase alkylate product.

7. The alkylation system of claim 6, wherein diffusion of the olefin into the reactor slurry is maintained at a rate that is lower than the rate of olefin reaction during alkylation.

8. The alkylation system of claim 1, wherein the slurry reactor operates at a reaction temperature of from about 40° C. to about 120° C.

9. The alkylation system of claim 1, wherein the slurry reactor further comprises one or more inter-cooling loops, each inter-cooling loop including a draw-off tray, at least one pump, and at least one cooler.

10. The alkylation system of claim 1, wherein the reactor liquid effluent stream is provided to a at least one first stage liquid solid separation device that separates catalyst from the liquid alkylate product and isobutane in the reactor liquid effluent stream.

11. An alkylation process for alkylation of isobutane with olefin to make alkylate for gasoline blending, the alkylation process comprising the steps of:
providing a slurry reactor that receives a reactor feed slurry including catalyst and liquid isobutane, an olefin feed that is in a vapor phase or vaporizes upon being injected into the slurry reactor, and a circulating reactor vapor stream;
providing the reactor feed slurry to the slurry reactor from a slurry vessel;
providing catalyst to the slurry vessel from a catalyst source;
providing a liquid isobutane feed stream to the slurry vessel from an isostripper;
combining the olefin feed and the circulating reactor vapor stream in the slurry reactor to form a combined reaction vapor; and
contacting the combined reaction vapor and the reactor feed slurry in the slurry reactor;
where an alkylation reaction occurs when the reaction vapor contacts the reactor feed slurry in the slurry reactor, and a reactor liquid effluent stream is produced, the reactor liquid effluent stream including catalyst, isobutane, and a liquid alkylate product.

12. The alkylation process of claim 11, wherein the catalyst source is a catalyst regenerator.

13. The alkylation process of claim 12, wherein the catalyst in the reactor feed slurry comprises regenerated catalyst from the catalyst regenerator.

14. The alkylation process of claim 11, wherein the reactor feed slurry comprises catalyst in an amount from about 2% by weight of the slurry to about 25% by weight of the slurry.

15. The alkylation process of claim 11, the slurry reactor including a plurality of distillation trays having space between each tray, and a plurality of distributors located in a space below at least a first distillation tray and above at least a second distillation tray, wherein the process further comprises the steps of:
dividing the olefin feed into a plurality of olefin injection streams; and
injecting the plurality of olefin injection streams into the slurry reactor through the distributors.

16. The alkylation process of claim 15, wherein the combined reaction vapor bubbles up through the first distillation tray, and the alkylation reaction occurs on the first distillation tray wherein olefin from the combined reaction vapor diffuses into the reactor slurry, reacts with the liquid isobutane in the presence of the catalyst, and produces a liquid phase alkylate product.

17. The alkylation process of claim 16, wherein diffusion of the olefin into the reactor slurry is maintained at a rate that is lower than the rate of olefin reaction during alkylation.

18. The alkylation process of claim 11, wherein the slurry reactor operates at a reaction temperature of from about 40° C. to about 120° C.

19. The alkylation process of claim 11, wherein the slurry reactor further comprises one or more inter-cooling loops, each inter-cooling loop including a draw-off tray, at least one pump, and at least one cooler.

20. The alkylation process of claim 11, the process further comprising the step of:
providing the reactor liquid effluent stream to a at least one first stage liquid solid separation device that separates catalyst from the liquid alkylate product and isobutane in the reactor liquid effluent stream.
